(19) United States
(12) Patent Application Publication
(10) Pub. No.: US 2026/0151251 A1
(43) Pub. Date: Jun. 4, 2026

(54) Neck Brace

(71) Applicant: YUNJIE MIDICAL AND HEALTH TECHNOLOGY (NANTONG) CO., LTD., Nantong (CN)

(72) Inventor: Jiang Wang, Beijing (CN)

(21) Appl. No.: 19/177,416

(22) Filed: Apr. 11, 2025

(30) Foreign Application Priority Data
Sep. 14, 2024 (CN) 202411292532.7
Sep. 30, 2024 (CN) 202411389241.X
Dec. 27, 2024 (CN) 202411952260.9
Dec. 27, 2024 (CN) 202423262616.4

(51) Int. Cl. A61F 5/055

(52) U.S. Cl. CPC A61F 5/055

(57) ABSTRACT

A neck brace device for wearing on a user's neck includes an adjustment platform for surrounding the neck of the user, wherein the adjust platform further includes a jaw support device. The jaw support device is disposed on the top portion of the adjustment platform for supporting the jaw of the user. The jaw support device is adjustable in a horizontal direction to adapt the width of the user's jaw.

### BACKGROUND OF THE PRESENT INVENTION

### Field of Invention

The present invention relates to physiotherapy device, and more particularly to a neck brace.

### Description of Related Arts

Individuals who work with their heads lowered for prolonged periods are prone to developing cervical spine issues. As a critical medical support device, the neck brace provides stability and protection to the cervical spine by restricting neck movement, thereby promoting recovery and reducing the risk of further injury. Proper selection of the neck brace's size and model is essential to ensure it conforms adequately to the user's neck. Currently, neck braces are generally available in limited standard sizes, such as large, medium, and small. These sizes are typically determined based on samples from many users to accommodate most individuals; however, they are not customized for each specific user. As a result, users may only achieve a relatively good fit rather than a perfect one. Even within the same size category, some users may find the neck brace fits well, while others may find it inadequate, thus affecting the device's effectiveness. Given that neck dimensions vary in length and thickness among individuals, there is a need for a neck brace capable of holistic adjustment to fit varying neck sizes. However, no such product currently exists in the market.

From the manufacturer's perspective, producing neck braces in multiple sizes necessitates the creation of different molds. While an increase in size options benefits more users, it also raises production costs due to the need for additional molds. Therefore, from a manufacturing standpoint, the ideal solution would be a single adjustable model that meets the needs of many users. For consumers, such a product would simplify the selection process and reduce decision-making effort. However, this type of neck brace is currently unavailable in the market, creating a significant need for a product that can address these challenges and meet market demands.”

### SUMMARY OF THE PRESENT INVENTION

An advantage of the present invention is to provides a neck brace, wherein the neck brace provides adjustment of the width of the jaw.

Another advantage of the present invention is to provide a neck brace, wherein the neck brace provides adjustment of the width of the neck.

Another advantage of the present invention is to provide a neck brace, wherein the neck brace provides adjust both the width and length of the neck.

Another advantage of the present invention is to provide a neck brace, wherein the neck brace provides adjust the angle of the jaw.

Another advantage of the present invention is to provide a neck brace, wherein the neck brace provides overall neck adjustment.

Another advantage of the present invention is to provide a neck brace, wherein the neck brace provides an adjustment platform configured to adjust the jaw width.

Another advantage of the present invention is to provide a neck brace, wherein the neck brace provides an adjustment platform configured to adjust the width of the lower portion of the neck.

Another advantage of the present invention is to provide a neck brace, wherein the adjustment platform is configured to adjust the neck width by adjusting both the jaw width and the width of the lower neck.

Another advantage of the present invention is to provide a neck brace, wherein the adjustment platform is configured to reduce the jaw width and drive the reduction of the lower neck width, thereby enhancing operational convenience.

Another advantage of the present invention is to provide a neck brace, wherein the adjustment platform is configured to adjust the jaw width via rotational operation, which further adjusts the width of the lower portion of the neck.

Another advantage of the present invention is to provide a neck brace, wherein the adjustment platform provides an upper section configured to adjust the jaw width and a lower section configured to adjust the width of the lower portion of the neck.

Another advantage of the present invention is to provide a neck brace, wherein the adjustment platform adjusts the neck width by providing rotational adjustment at the lower section of the adjustment platform.

Another advantage of the present invention is to provide a neck brace, wherein the adjustment platform provides the height adjustment and the jaw angle adjustment to adapt the length of the user's neck.

Another advantage of the present invention is to provide a neck brace, wherein the adjustment platform provides horizontal adjustment.

Another advantage of the present invention is to provide a neck brace, wherein the adjustment platform provides vertical adjustment.

Another advantage of the present invention is to provide a neck brace, wherein the adjustment platform comprises an adjustable base configured to provide horizontal adjustment.

Another advantage of the present invention is to provide a neck brace, wherein the adjustment platform comprises two adjustment devices configured to provide vertical adjustment.

Another advantage of the present invention is to provide a neck brace, wherein the adjustment platform comprises two mounting platforms for attaching the adjustment devices, each forming a cushioning arrangement.

Another advantage of the present invention is to provide a neck brace, wherein the adjustment device comprises a distance adjustment device and an angle adjustment device configured to cooperate in providing vertical adjustment.

Another advantage of the present invention is to provide a neck brace, wherein the adjustable base comprises a fixing portion that defines the width of the base.

Another advantage of the present invention is to provide a neck brace, wherein the adjustable base comprises two shoulder supporters that are rotatably connected to adjust the angle between the two shoulder supporters.

Another advantage of the present invention is to provide a neck brace, wherein the angle adjustment device is adjacent to the distance adjustment device and is configured to lock the angle during the extension of the distance adjustment device.

Another advantage of the present invention is to provide a neck brace, wherein the adjustment platform comprises a jaw support device, and the angle adjustment device adjusts the angle of the jaw support device.

Another advantage of the present invention is to provide a neck brace, wherein the jaw support device provides adjustable width to adapt the jaw width of the user.

Another advantage of the present invention is to provide a neck brace, wherein the jaw support device comprises a knob element configured to adjust the distance between the two ends of the jaw support device.

Another advantage of the present invention is to provide a neck brace, wherein the neck width is adjusted by adjusting the two shoulder supporters and the jaw support device.

Additional advantages and features of the invention will become apparent from the description which follows, and may be realized by means of the instrumentalities and combinations particularly pointing out in the appended claims.

To achieve at least one of the mentioned advantages or other objectives, according to one aspect of the present invention, the present invention provides a neck brace for wearing around the neck of a user. The neck brace comprises:
an adjustment platform for surrounding the neck of the user, wherein said adjust platform further comprises a jaw support device, wherein the jaw support device is disposed on the top portion of said adjustment platform for supporting the jaw of the user, wherein said jaw support device is adjustable in a horizontal direction to adapt the width of the user's jaw.

According to an embodiment of the present invention, wherein said neck brace device further comprises an adjustable base disposed below the jaw support device wherein the adjustable base is configured to be adjusted in a horizontal direction to adapt to the width of the user's neck, wherein horizontal adjustment of said jaw support device to reduce said jaw support device's width guides corresponding adjustment of said adjustable base to reduce the width of said adjustable base.

According to an embodiment of the present invention, wherein the adjustable base further comprises two shoulder support members adjustably connected, wherein a wearing cavity is defined between said two shoulder support members to receive the user's neck, wherein when said jaw support device is adjusted in a horizontal direction to reduce said jaw support device's width, said two shoulder support members are guided to move toward each other to reduce said width of the wearing cavity.

According to an embodiment of the present invention, wherein said two shoulder support members are pivotally connected, wherein when said jaw support device is adjusted in a horizontal direction to reduce the width of the jaw support device, said two shoulder support members are guided to pivot toward the user's neck.

According to an embodiment of the present invention, wherein the adjustment platform further comprises two adjustment devices, the two adjustment devices being separately disposed between said tow shoulder support members and the jaw support device, configured to adjust a distance between the two shoulder support members and the jaw support device and an angle of the jaw support device, wherein the jaw support device is rotatable toward the two shoulder support members to support the user's jaw in a head-down posture.

According to an embodiment of the present invention, wherein the jaw support device further comprises: two side support members, said two side support members being separately rotatably connected to said two adjustment devices, such that said two adjustment devices adjust and angel of said jaw support device; and a jaw adjustment member, opposite ends of said jaw adjustment member being connected to respective ends of the two side support members, the jaw adjustment member is adjusted in a horizontal direction to adapt a width of the user's jaw, wherein, by adjusting the jaw adjustment member to reduce the jaw adjustment member's width, the two side support members are driven to move toward the user's neck, thereby driving the two adjustment devices to move toward said user's neck, and further causing the two shoulder support members to move toward the user's neck.

According to an embodiment of the present invention, wherein the jaw adjustment member further comprises a support unit and an adjustment unit, wherein the adjustment unit is movably connected to the support unit, wherein the support unit is configured to conform to said user's jaw, and wherein the length of the adjustment unit is adjustable to adapt the width of the user's jaw.

According to an embodiment of the present invention, wherein the adjustment unit further comprises:
an adjustment knob, wherein one end of the knob is provided with a gear;
two adjustment straps, each of the adjustment straps having a toothed side with a plurality of teeth formed thereon to define a toothed belt, wherein the adjustment knob is disposed between the two adjustment straps, wherein the toothed sides of the toothed sides of the two adjustment straps face each other, and wherein the gear of the adjustment knob is engaged with the toothed belts of the two adjustment straps, such that the width of the adjustment unit is adjustable by changing the relative positions of the two adjustment straps.

According to an embodiment of the present invention, wherein the adjustment unit is selected from a parallel displacement mechanism and an elastic device.

According to an embodiment of the present invention, wherein the adjustable base further comprises an adjustment portion, the two shoulder support members are separately connected to the adjustment portion, wherein the adjustment portion further comprises a rotation shaft and a coupling member, the coupling member is sleeved over the rotation shaft such that the two shoulder support members are rotatable about the rotation shaft.

According to an embodiment of the present invention, wherein the adjustable base further comprises an adjustment portion, the two shoulder support members are separately rotatably connected to the adjustment portion, wherein the adjustment portion further comprises two rotation shafts and two rotation shafts and two coupling members, each of the coupling members is sleeved over a respective rotation shaft such that the two shoulder support members are rotatably connected to the adjustment portion.

According to an embodiment of the present invention, wherein the rotation shaft comprises a first limiting member disposed on an outer side of a bottom portion of the first limiting member, and the coupling member comprises a second limiting member disposed on an outer side of a bottom portion of the coupling member, wherein a clearance is defined between two ends of the first limiting member and the second limiting member to form an angle-limiting mechanism that restricts a rotation angle of the two shoulder adjustment devices.

According to an embodiment of the present invention, wherein the adjustment base further comprises an adjustment portion, where the adjustment portion is selected from a parallel linkage mechanism, a ratchet mechanism, and an elastic band.

According to an embodiment of the present invention, wherein the adjustment device further comprises a distance adjustment device and an angle adjustment device, wherein the angle adjustment device is configured to adjust an angle of said jaw support device, and wherein said angle adjustment device locks the jaw support device in position to prevent rotation of the jaw support device during vertical adjustment by said distance adjustment device.

According to an embodiment of the present invention, wherein the distance adjustment device further comprises a knob, an adjustment rack, and adjustment bracket, and an adjustment device housing, wherein the knob and the adjustment rack are mounted on the adjustment bracket, wherein the adjustment bracket is mounted on the mounting bracket, and wherein the adjustment bracket is adapted within the adjustment device housing.

According to an embodiment of the present invention, wherein said angle adjustment device, further comprises a fixed portion, a rotation portion, and an angle-limiting portion, wherein said fixed portion and said rotation portion are disposed opposite each other, wherein one end of said angle-limiting portion is movable between the interior of said fixed portion and the interior of the rotation portion, wherein when the said one end of said angle-limiting portion is positioned between said fixed portion and said rotation portion, said angel adjustment device is in a locked state such that the position of said jaw support device is fixed, wherein when said one end of said angle-limiting portion is positioned within said rotation portion, said angle adjustment device is an unlocked state, allowing said jaw support device to be adjusted to a suitable angle.

According to an embodiment of the present invention, wherein the angle-limiting portion comprises an angle-limiting member and an elastic element, wherein the elastic element is disposed within the rotation portion, and a bottom end of the angle-limiting member is in contact with said elastic element, wherein when said elastic element is not compressed, the angle-limiting member is positioned between the fixed portion and the rotation portion to prevent said rotation portion from rotating, wherein when said elastic element is compressed, said angle-limiting member is displaced into said rotation portion to allow said rotation portion to move.

According to an embodiment of the present invention, wherein the neck brace further comprises a rear neck support portion, wherein the rear neck support portion is detachably connected to said two adjustment devices to conform to a rear neck of the user by adjusting the rear neck support portion.

According to another aspect of the present invention, the present invention provides a neck brace for wearing around the neck of a user. The neck brace comprises:
an adjustment platform configured to be worn around the neck of the user, wherein the adjustment platform comprises a top portion and a bottom portion, the width of the top portion and the bottom portion is adjusted separately, wherein a reduction in the width of the bottom portion is driven by the reduction in the width of the top portion, for reducing the width of the adjustment platform, such that the top portion and the bottom portion are fit for the neck of the user, wherein the adjustment platform further comprises,
a jaw support device configured to the top portion of the adjustment platform, wherein the jaw support device is modified the width of the jaw of the user through perform the horizontal adjustment of the jaw support device; and
an adjustable base configured to the bottom portion of the adjustment platform, wherein the adjustable base provide

According to an embodiment of the present invention, the adjustment platform further comprises an adjustable base disposed below the jaw support device, the adjustable base being configured for horizontal adjustment to adapt the width of the user's neck, wherein when the jaw support device is adjusted horizontally to reduce the width of the jaw support device, the adjustable base is guided to correspondingly reduce the width of the adjustable base.

According to an embodiment of the present invention, the adjustable base further

comprises two shoulder support members the two shoulder support member are adjustably connected, wherein the shoulder supporters define a wearing cavity configured to adapt the neck of the user. When the jaw support device is adjusted horizontally to reduce the width of the jaw support device, the shoulder support members are guided to move toward each other, thereby reducing the width of the wearing cavity.

According to an embodiment of the present invention, the two shoulder support members are pivotally connected the jaw support device separately. When the jaw support device is adjusted horizontally to reduce the width of the jaw support device, the shoulder support members are guided to close to the user's neck in a rotation method.

According to an embodiment of the present invention, the adjustment platform further comprises two adjustable devices disposed between the shoulder support members and the jaw support device. The adjustable devices is adjust the distance between the shoulder support members and the jaw support device, as well as the angle of the jaw support device. The jaw support device is able to be rotated toward the shoulder support members to support the user in a head-down posture.

According to an embodiment of the present invention, the jaw support device further comprises:
two side support members, each rotatably connected to the adjustable device for allowing the adjustable device to adjust the angle of the jaw support device; and
a jaw adjustable element, with two ends of the jaw adjustment connected to the side support members separately, wherein the jaw adjustment component is horizontally adjustable to fit the width of the user's jaw, wherein adjusting the jaw adjustable element to reduce the jaw adjustable element's width so as to drive the side support members closer to the user's neck, thereby moving the adjustable device and shoulder support members toward the user's neck.

According to an embodiment of the present invention the jaw adjustable element further comprises:
a support unit configured to conform to the user's jaw; and
an adjustment unit movably connected to the support unit, wherein the adjustment unit has an adjustable length to adapt the width of the user's jaw.

According to an embodiment of the present invention, the adjustment unit further comprises:
an adjustment knob element, one end of the adjustment knob element is equipped with a gear; and
two adjustment straps, each adjustment strap having a set of teeth formed on one side for defining a toothed area, wherein the adjustment knob element is disposed between the two adjustment straps, the toothed sides of the adjustment strap are arranged in opposition such that the gear of the adjustment knob engages with the toothed area of the two adjustment straps. By rotating the adjustment knob, the relative position of the two adjustment straps is adjusted for adjusting the width of the width of the adjustment unit.

According to an embodiment of the present invention, the adjustment unit is selected from a parallel displacement mechanism or an elastic device.

According to an embodiment of the present invention, the adjustable base further comprises two shoulder support members and an adjustment portion, each end of the two shoulder support members connected to the adjustment portion, wherein the adjustment portion allows for the distance between the shoulder support members to be adjusted.

According to an embodiment of the present invention, the adjustment portion further comprises a rotating shaft and a coupling member. The coupling member is sleeved over the rotation shaft, such that rotational movement of the coupling member adjusts the distance between the two shoulder support devices.

According to an embodiment of the present invention, the adjustment portion further comprises two rotation shafts and two coupling members, each of the coupling members being sleeved over a respective one of the rotation shafts, such that rotational movement of the coupling members adjusts a distance between the two shoulder support members.

According to an embodiment of the present invention, the adjustment section is selected from a parallel motion structure, an elastic device, or a ratchet mechanism.

According to an embodiment of the present invention, the adjustment mechanisms further comprise a distance adjustment device and an angle adjustment device, wherein the angle adjustment device is disposed on one side of the distance adjustment device, wherein the distance adjustment device comprises:
an adjustment knob, an adjustment rack, an adjustment bracket, and an adjustment housing, wherein the knob and adjustment rack are mounted on the adjustment bracket, and the adjustment bracket is mounted within the adjustment device housing.

According to an embodiment of the present invention, the angle adjustment device further comprises:
a fixed portion,
a rotating portion, and
an angle-limiting portion, wherein the fixed portion and rotating portion are positioned opposite to each other, and one end of the angle-limiting portion is configured to moves between the interior of the fixed portion and the rotating portion. When the end of the angle-limiting portion is positioned between the fixed portion and rotating portions, the angle adjustment device is in a locked state, thereby fixing the position of the jaw support device. When the end of the angle-limiting portion is positioned within the rotating portion, the angle adjustment device is in an unlocked state, thereby allowing the jaw support device to be adjusted to a desired angle.

According to an embodiment of the present invention, the angle-limiting portion comprises an angle-limiting member and an elastic element, wherein the elastic element is disposed within the rotating portion, and the bottom of the angle-limiting member contacts the elastic element, wherein when the elastic element is not compressed, the angle-limiting member is positioned between the fixed and rotating portions, preventing the rotating portion from moving, when the elastic element is compressed, the angle-limiting member moves to the rotating portion, enabling rotation.

According to an embodiment of the present invention, the neck brace further comprises a rear neck support portion that is detachably connected to the adjustment platform, wherein when the rear neck support portion conforms to the user's neck, the sides of the adjustment platform gradually conform to the user's neck, thereby providing secondary adjustment.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a schematic diagram illustrating a neck brace according to an embodiment of the present application.

FIG. 2 is an exploded view of a neck brace according to an embodiment of the present application.

FIG. 3A to 3B illustrate an adjustable base of a neck brace according to an embodiment of the present application, showing the adjustment process of the adjustable base.

FIG. 4A to 4C illustrate an adjustment mechanism of a neck brace according to an embodiment of the present application, showing the adjustment process of the adjustment mechanism.

FIG. 5 is a schematic diagram illustrating a jaw support device of a neck brace according to an embodiment of the present application.

FIG. 6A to FIG. 6B illustrate a neck brace according to an embodiment of the present application, showing the width adjustment of the neck brace.

FIG. 7A to FIG. 7B illustrate a jaw support device of a neck brace according to an embodiment of the present application, showing the height adjustment of the neck brace.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

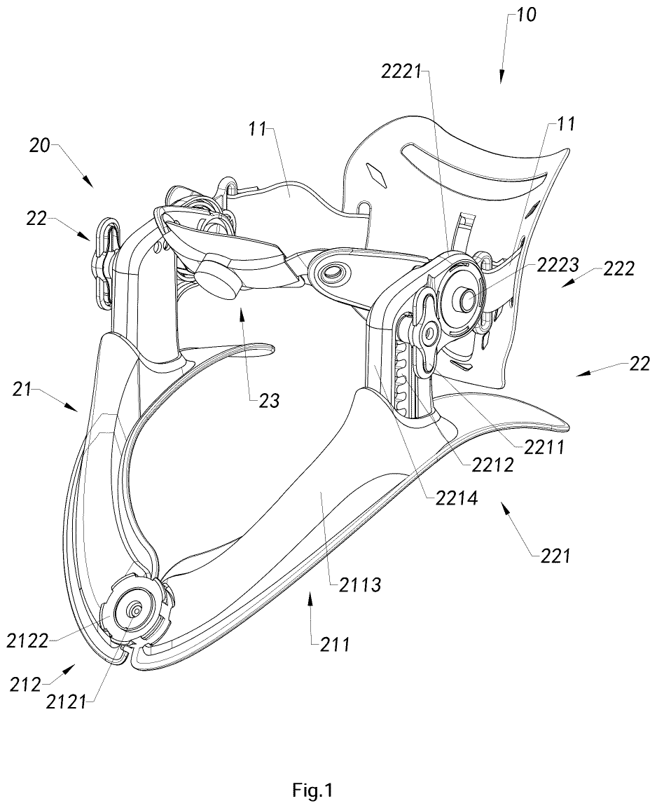

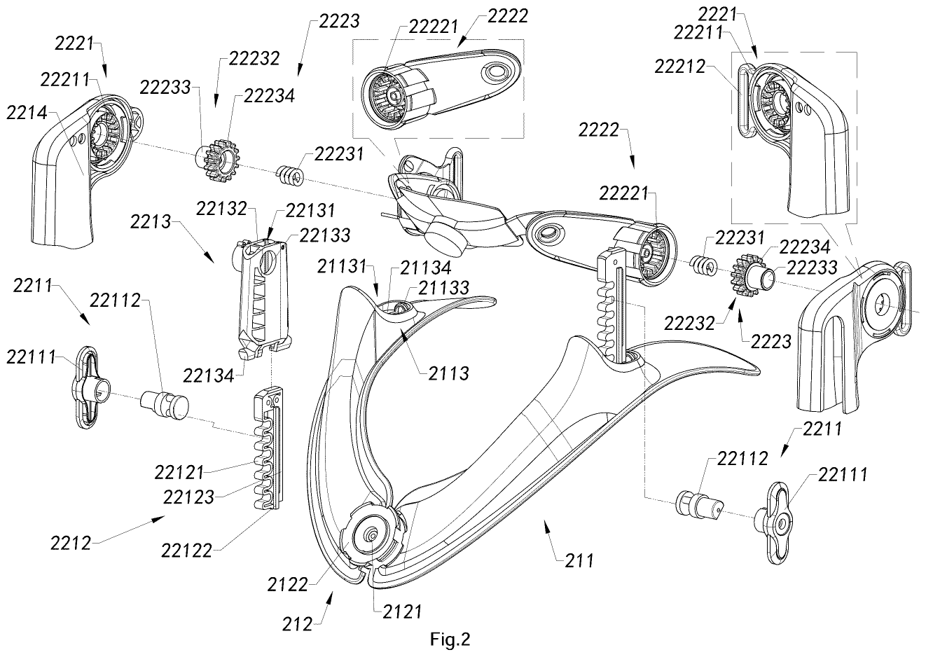

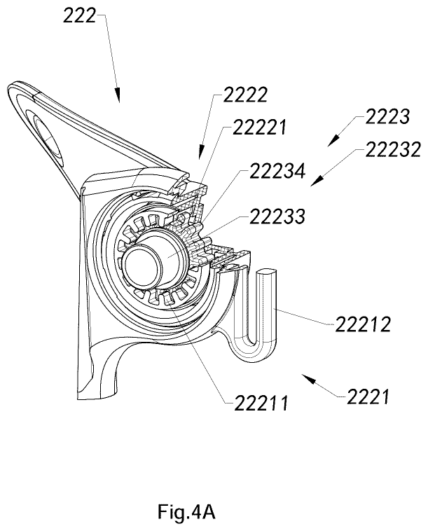

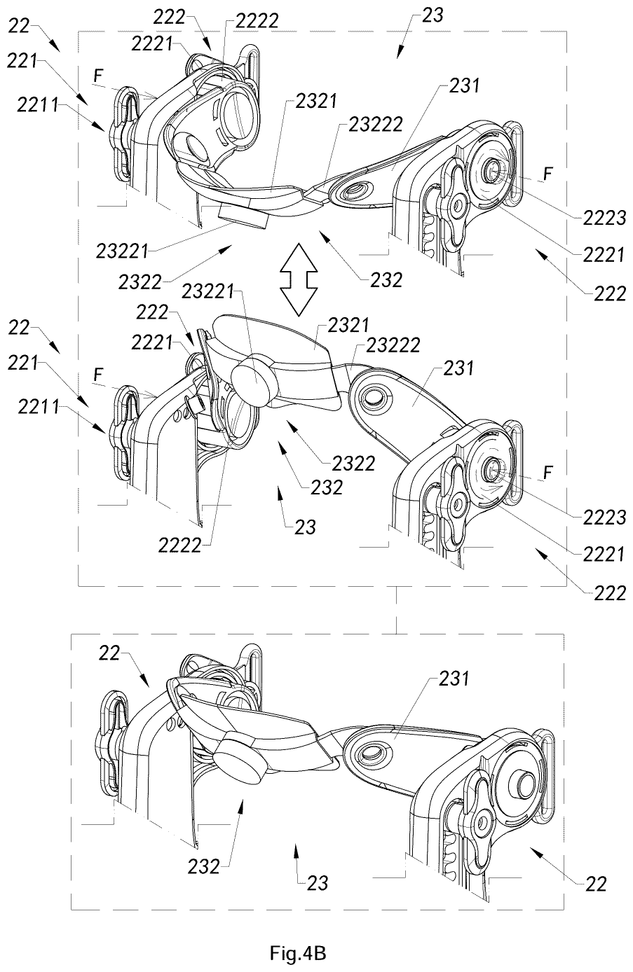

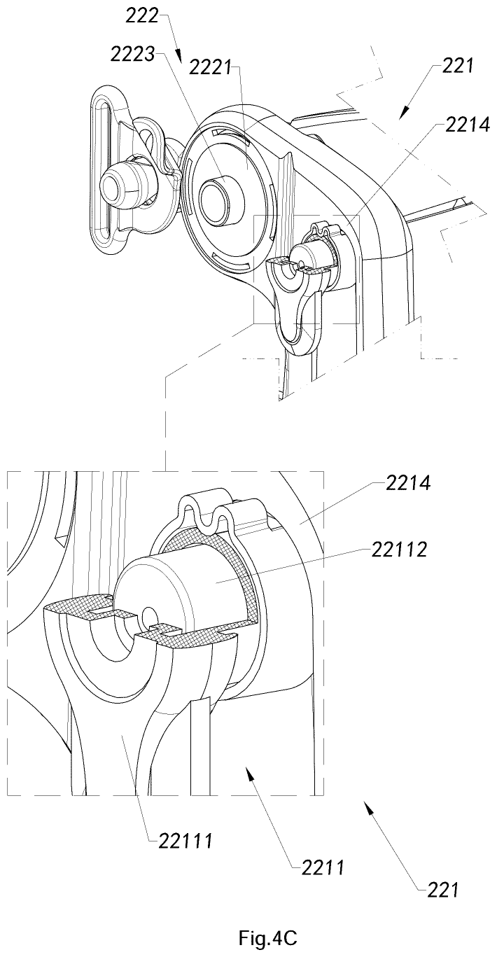

Referring to FIGS. 1 to 5, a neck brace device according to the present invention comprises an adjustment platform 20, which further defines a wearing cavity 24. The wearing cavity 24 is configured to receive the user's neck when the adjustment platform 20 is worn, such that the adjustment platform 20 surrounds the user's neck. The wearing cavity 24 further defines a wearing opening 241, through the waring cavity 24 the user wear the adjustment platform 20. When the user wears the adjustment platform 20 around the user's neck, the adjustment platform 20 is adjustable to adapt to the user's neck. Specifically, the adjustment platform 20 adjusts the width of the wearing cavity 24 to fit the width of the user's neck, thereby securing the user's neck.

The neck brace device further comprises a rear neck support portion 10, which is detachably connected to the adjustment platform 20. After the adjustment platform 20 is worn on the user's neck, the rear neck support portion 10 is positioned against the rear side of the user's neck and then secured to the adjustment platform 20. In other words, the rear neck support portion 10 conforms to the rear portion of the user's neck to provide rear portion of the user's neck support and further stabilize the user's neck. Once the rear neck support portion 10 is secured, the adjustment platform 20 and the rear neck support portion 10 collectively stabilize the user's neck. In a preferred embodiment, two adjustment straps 21 are respectively used to detachably connect opposing sides of the rear neck support portion 10 to corresponding sides of the adjustment platform 20.

According to a preferred embodiment of the present application, the user's neck has a certain length. Accordingly, in the present application, the adjustment of the neck width is achieved by separately adjusting the upper portion and lower portion of the user's neck. In a preferred embodiment of the present application, the width of the upper neck portion is adjusted by modifying the width of the user's mandible. The top portion of the adjustment platform 20 provides a mechanism for adjusting the width of the jaw to thereby effect adjustment of the upper neck portion. The bottom portion of the adjustment platform 20 provides a mechanism for adjusting the width of the lower neck portion. By separately adjusting the width of the upper and lower portions of the user's neck, the overall width of the user's neck can be effectively adjusted. Furthermore, the adjustment platform 20 is configured to adjust the upper and lower portions of the user's neck independently, thereby accommodating a wider variety of neck types among users. For example, in certain user cases, the upper portion of the user's neck may be wider than the lower portion, or the lower portion may be wider than the upper portion. In other words, the adjustment platform 20, according to a preferred embodiment of the present application, is capable of adapting to instances where the width of the upper portion of the user's neck differs from that of the lower portion.

The adjustment platform 20 further provides an adjustment for the angle of the jaw. After the angle of the user's jaw is adjusted, the adjustment platform 20 further enables vertical adjustment to position the user's neck at a proper position. In the proper position the adjustment platform 20 secures the user's neck and further enables traction of the user's cervical spine. In a preferred embodiment of the invention, the proper position refers to a state in which the user's jaw is slightly tilted upward. The adjustment platform 20 adjusts the upward tilt angel of the user's jaw to bring the jaw into a suitable position. Thereafter, the adjustment platform 20 is adjusted in the vertical direction such that the user's neck is supported and further subjected to traction in the appropriate position. It is worth mentioning that most users work in a forward head posture. Therefore, a slight upward tilt of the jaw helps the cervical spine extend and restores its natural curvature. By using the adjustment platform 20 for vertical adjustment, the user is secured in an extended posture, thereby supporting the user's neck. Furthermore, this adjustment achieves a traction effect on the user's cervical spine. To facilitate vertical adjustment of the adjustment platform 20 and ensure that the user's neck is supported in an appropriate position, the adjustment platform 20 is provided with a cushioning structure. This cushioning structure allows the adjustment platform 20 to be positioned comfortably during adjustment while providing effective support to the user's neck.

The adjustment platform 20 further comprises an adjustable base 21, two adjustment devices 22, and a jaw support device 23. The two adjustment devices 22 are respectively mounted on opposite sides of the adjustable base 21. The jaw support device 23 is adjustably connected to the two adjustment devices 22. Specifically, the bottom portions of the two adjustment devices 22 are mounted on opposite sides of the adjustable base 21, while the top portions of the two adjustment devices 22 are connected to opposite sides of the jaw support device 23. The adjustable base 21 defines the wearing cavity 24 in the central portion of the adjustable base 21. The jaw support device 23 is positioned at the top of the adjustment platform 20, and the adjustable base 21 is positioned at the bottom of the adjustment platform 20. The jaw support device 23 provides horizontal adjustment to adapt to the width of the user's jaw, while the adjustable base 21 provides horizontal adjustment to adapt to the width of the lower portion of the user's neck. When the jaw support device 23 is adjusted to reduce the width of the jaw support device 23, the adjustable base 21 moves closer to the user's neck along with the movement of the jaw support device 23, thereby reducing the width of the adjustable base 21. Specifically, as the jaw support device 23 is adjusted to reducing the jaw support device 23's width, the jaw support device 23 moves closer to the user's neck, thereby driving the two adjustment devices 22 toward the user's neck, which in turn drives the two adjustment devices 22 to move toward the user's neck, thereby causing the adjustable base 21 to move closer to the user's neck, thereby causing the adjustable base 21 to move closer to the user's neck and reducing the width of the adjustable base 21.

In a preferred embodiment of the present invention, after the adjustment platform 20 is worn around the user's neck, the adjustable base 21 extends from the user's shoulders along the chest to the front portion of the chest of the user. Preferably, the adjustable base 21 extends toward and conforms to the user's back. The top ends of the two adjustment devices 22 are adjustably connected to respective sides of the jaw support device 23. Preferably, the end portions of the two sides of the jaw support device 23 are adjustably mounted to the top ends of the respective adjustment devices 22. By adjusting the angular position of the jaw support device 23 via the two adjustment devices 22, the user's cervical spine can be placed in a comfortable posture by slightly tilting the user's jaw upward. Preferably, the jaw support device 23 is adjustable in a rotational manner to support the user's jaw at the desired angle. Once the user's jaw is positioned in a comfortable posture, the two adjustment devices 22 lock the jaw support device 23 in place, thereby restricting the jaw support device's 23 rotation. It's worth mentioning that, when the two adjustment devices 22 are further adjusted in the vertical direction, the opposing sides of the adjustable base 21 move slightly away from the user's neck to form a cushioning space 25 between the user's neck and the jaw support device 23. The cushioning space 25 facilitates two main functions: first, the cushioning space 25 allows the adjustment mechanisms 22 to position the jaw support device 23 at the proper location; second, the cushioning space 25 enhances user comfort during the adjustment process. In contrast, existing cervical traction devices lack such a cushioning space, resulting in the user's neck being positioned in a suboptimal or less-than-proper posture, the inability to achieve a proper position is due to the tight contact between the existing traction device and the user's neck, which prevents further movement toward the ideal position. As a result, during adjustments from a suboptimal to a proper position, the adjustment device 22 of conventional devices press directly against the user's neck skin, thereby increasing friction and causing discomfort or even pain. Consequently, users of existing cervical traction devices cease adjustments at a suboptimal position, reducing the effectiveness of the devices and failing to achieve the desired therapeutic outcome. The neck brace device of the present invention provides a cushioning space 25, which minimizes friction between the adjustment device 23 and the user's neck skin. This facilitates smooth transition from a suboptimal to an optimal position and significantly improves user experience by enhancing comfort during adjustment. Preferably, after adjustment to the proper position is completed, both sides of the adjustable base 21 move closer to the user's neck to conform tightly to the neck for improved support.

The adjustable base 21 further comprises two shoulder support members 211 and an adjustment portion 212. Opposite ends of the two shoulder support members 211 are respectively connected to the adjustment portion 212. A wearing cavity 24 is defined between the two shoulder support members 211. The adjustment portion 212 is configured to rotatably adjust the angle between the two shoulder support members 211.

The adjustment section 212 further comprises a rotating shaft 2121 and a coupling member 2122. The coupling member 2122 is sleeved over the rotation shaft 2121 such that the coupling member 2122 is rotatable around the rotation shaft 2121. The rotation shaft 2121 is disposed at one end of one of the shoulder support members 211, and the coupling member 2122 is disposed at one end of the other shoulder support member 211. Specifically, the rotation shaft 2121 is centrally located at a shaft end 2111 of one of the shoulder support members 211. Preferably, the diameter of the rotation shaft 2121 is smaller than the width of the shaft end 2111. The coupling member 2122 is centrally located at a coupling end 2112 of the other shoulder support member 211. Preferably, the diameter of the coupling end 2112 is smaller than the middle portion of the rotating shaft 2121.

The adjustment portion 212 further comprises an angle-limiting mechanism 2123. The angle-limiting mechanism 2123 is disposed on the rotating shaft 2121 and the coupling member 2122. Specifically, the angle-limiting mechanism 2123 comprises a first limiting element 21231 and a second limiting element 21232. The first limiting element 21231 is positioned on the outer bottom side of the rotating shaft 2121, and the second limiting element 21232 is positioned on the outer bottom side of the coupling member 2122. When the coupling member 2122 is sleeved over the rotating shaft 2121, the first limiting element 21231 and the second limiting element 21232 are aligned on the same plane, with a gap defined therebetween. The gap defines the rotation range of the coupling member 2122. When the other shoulder support member 211 approaches the one shoulder support member 211, the coupling member 2122 rotates about the rotation shaft 2121 until the first limiting element 21231 contacts the second limiting element 21232, thereby restricting further rotation of the other shoulder support member 211.

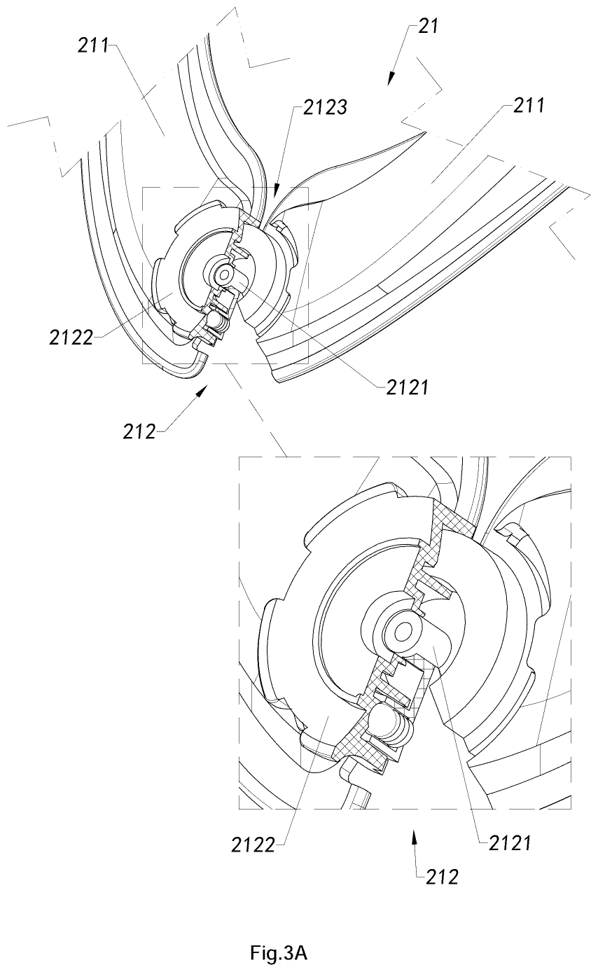

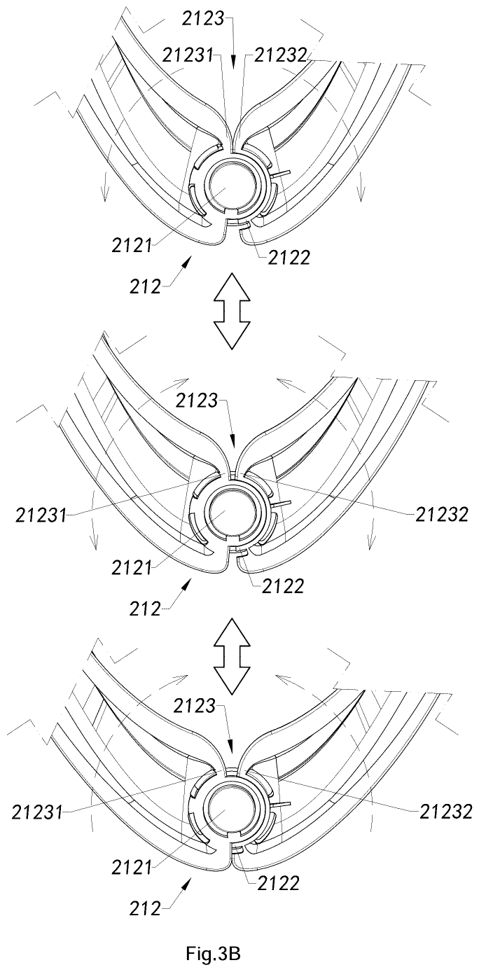

As shown in FIG. 3, the shoulder support members 211 have a curved configuration. Specifically, each of the shoulder support members 211 extends downward so as to conform to the user's shoulders and chest. Furthermore, the shoulder support members 211 extend from the user's shoulders toward the user's chest. Preferably, when the shoulder support members 211 extend to the user's chest, the shoulder support members 211 converge toward the center of the user's chest, such that the two shoulder support members 211 are connected at the middle portion of the chest. In a preferred embodiment of the present invention, the adjustment section 212 is disposed at the middle portion of the user's chest. The rotating shaft end 2121 of one of shoulder support members 211 and the coupling end 2112 of the other shoulder support member 211 are connected at the middle portion of the user's chest. Preferably, the adjustable base is U-shaped.

The shoulder support member 211 further comprises a mounting bracket 2113. The mounting bracket 2113 is secured to a curved surface to form a mounting portion 21131 on the curved surface for receiving the adjustment device 22. Preferably, a bottom portion of the adjustment device 22 is insertable into the mounting portion 21131. The mounting portion 21131 further comprises a guide wall 21132. The guide wall 21132 is configured to guide the adjustment device 22 to be mounted vertically on the mounting bracket 2113. The guide wall 21132 is disposed on a side of the mounting bracket 2113 proximal to the adjustment portion 212. Additionally, the guide wall 21132 maintains the vertical orientation of the adjustment device 22. Furthermore, the mounting bracket 2113 directs pressure received by the adjustment device 22 from the user's head toward the user's shoulders and chest, thereby relieving pressure from the user's shoulders. In other words, the mounting bracket 2113 functions to distribute applied forces, thereby enhancing the comfort of the neck brace device during use.

The adjustment mechanism 22 further comprises a distance adjustment mechanism 221 and an angle adjustment mechanism 222, wherein the angle adjustment mechanism 222 is positioned on one side of the distance adjustment mechanism 221. The bottom portion of the distance adjustment mechanism 221 is mounted to the mounting section 21131, allowing the distance adjustment mechanism 221 to be adjustably secured. The angle adjustment mechanism 222 is adjustably connected to the jaw support device 23 to adjust the angle of the jaw support device 23. The distance adjustment mechanism 221 further comprises a knob 2211, an adjustment rack 2212, an adjustment bracket 2213, and an adjustment mechanism housing 2214. The knob 2211 and the adjustment rack 2212 are installed on the adjustment bracket 2213. The knob 2211 adjusts the vertical movement of the adjustment rack 2212 within the adjustment bracket 2213. The adjustment rack 2212 further comprises a set of teeth 22121, which are located on one side of the adjustment rack 2212. Additionally, the adjustment rack 2212 comprises a limiting bar 22122. The limiting bar 22122 is positioned at the middle portion of the adjustment rack 2212. Preferably, the limiting bar 22122 is located at the rear portion of the set of teeth 22121. The limiting bar 22122 restricts the vertical movement of the adjustment rack 2212. Preferably, the adjustment rack 2212 further includes a limiting groove 22123. The limiting groove 22123 is configured to match the adjustment bracket 2213, thereby constraining the adjustment rack 2212 to vertical movement within the adjustment bracket 2213.

The adjustment device 22 further comprises a distance adjustment device 221 and an angle adjustment device 222. The angle adjustment device 222 is disposed on one side of the distance adjustment device 221. A bottom portion of the distance adjustment device 221 is mounted to the mounting portion 21131 such that the distance adjustment device 221 is adjustably secured thereto. The angle adjustment device 222 is adjustably coupled to the jaw support device 23, allowing adjustment of the angle of the jaw support device 23. The distance adjustment device 221 further comprises a knob element 2211, an adjustment rack 2212, an adjustment bracket 2213, and an adjustment housing 2214. The knob element 2211 and the adjustment rack 2212 are installed on the adjustment bracket 2213. Rotation of the knob element 2211 causes vertical movement of the adjustment rack 2212 within the adjustment bracket 2213. The adjustment rack 2212 further comprises a set of teeth element 22121 formed on one side thereof. The adjustment rack 2212 also comprises a limiting strip 22122 positioned at a central portion thereof. Preferably, the limiting strip 22122 is disposed on the rear side of the set of teeth 22121. The limiting strip 22122 defines the vertical movement range of the adjustment rack 2212. Preferably, the adjustment rack 2212 further comprises a limiting slot 22123 configured to correspond to the adjustment bracket 2213, such that vertical movement of the adjustment rack 2212 within the adjustment bracket 2213 is constrained.

The adjustment bracket 2213 further comprises at least one directional limiting slot 22131, which corresponds to the limiting strip 22122. That is, the limiting strip 22122 is embedded into the directional limiting slot 22131, such that the limiting strip 22122 is guided to move vertically along the directional limiting slot 22131. Preferably, the directional limiting slot 22131 is disposed on an inner side of the adjustment bracket 2213. The adjustment bracket 2213 further comprises a limiting member 22132, which is disposed inside the adjustment bracket 2213. The limiting member 22132 passes through the limiting slot 22123 to define a travel range of the adjustment rack 2212. The adjustment bracket 2213 further comprises a mounting cavity 22133. The mounting cavity 22133 is formed at a top portion of the adjustment bracket 2213 and configured to receive the knob element 2211.

The knob element 2211 further comprises an operation portion 22111 configured to facilitate manual operation of the knob element 2211 by a user. The knob element 2211 further includes a rotation shaft 22112 and an adjustment gear 22113. The adjustment gear 22113 is configured to engage with the set of teeth element 22121 of the adjustment rack 2212. By rotating the adjustment gear 22113 to engage different teeth element 22121, the adjustment rack 2212 is able to be driven to move in a vertical direction. The adjustment gear 22113 is disposed at an end of the rotation shaft 22112, and the rotation shaft 22112 is coupled to the operation portion 22111, such that rotation of the operation portion 22111 rotates the rotation shaft 22112. In a preferred embodiment, the adjustment gear 22113 further comprises two strip-shaped teeth element 22114 disposed along the edge of the adjustment gear 22113. The two strip-shaped teeth element 22114 are arranged opposite each other and simultaneously engage with the adjustment rack 2212. Preferably, the strip-shaped teeth element 22114 are arranged in an axial direction. The adjustment bracket 2213 further comprises at least one limiting shaft 22134, which is disposed at the bottom of the adjustment bracket 2213. The mounting portion 21131 of the mounting bracket 2113 further includes a mounting slot 21133 configured to receive the limiting shaft 22134. When the adjustment bracket 2213 is subjected to pressure from the neck, the adjustment bracket 2213 is capable of slight rotational movement to buffer the applied force. The limiting shaft 22134 is centrally located on the adjustment bracket 2213, and the mounting slot 21133 is centrally formed in the mounting portion 21131 as a recessed area. Preferably, two mounting surfaces 21134 are formed on respective sides of the mounting slot 21133. The two mounting surfaces 21134 correspond to the opposing bottom surfaces of the limiting shaft 22134 of the adjustment bracket 2213. Preferably, the limiting shaft 22134 is slightly elevated relative to the mounting slot 21133, thereby forming a cushioning gap between the bottom of the adjustment bracket 2213 and the bottom of the mounting portion 21131. This slight rotation of the adjustment bracket 2213 allows for pressure relief during use.

According to one embodiment of the present application, the adjustment device housing 2214 comprises an adjustment bracket receiving cavity 22141 configured to receive the adjustment bracket 2213. The adjustment device housing 2214 further comprises an adjustment knob channel 22142. A knob element 2211 extends through the adjustment knob channel 22142 to protrude outside of the adjustment device housing 2214. By rotating the knob element 2211, the user causes vertical movement of the adjustment rack 2212, thereby driving corresponding vertical movement of the adjustment device housing 2214 in unison with the adjustment rack 2212. During such vertical movement of the adjustment device housing 2214, the knob element 2211 remains disposed within the knob channel 22142. The adjustment device housing 2214 further comprises an adjustment bracket coupling portion 22143 disposed at an inner top side of the adjustment device housing 2214 and coupled to the adjustment bracket 2213. When the adjustment bracket 2213 moves upward in the vertical direction, it supports corresponding upward movement of the adjustment device housing 2214. When the adjustment bracket 2213 moves downward in the vertical direction, the adjustment device housing 2214 moves downward accordingly.

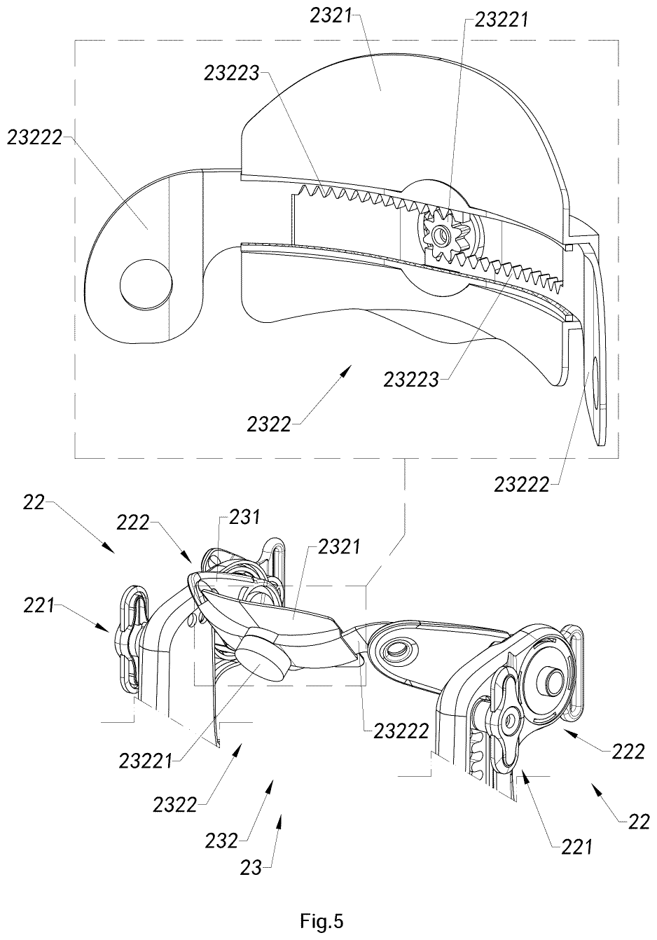

As shown in FIG. 5, the angle adjustment mechanism 222 is disposed on one side of the distance adjustment mechanism 221. Preferably, the angle adjustment device 222 further comprises a fixed portion 2221, a rotating portion 2222, and an angle-limiting portion 2223. The fixed portion 2221 and the rotating portion 2222 are arranged opposite to each other for defining a limiting cavity 2224. The angle-limiting portion 2223 is movable within the limiting cavity 2224 to control the rotation of the rotating portion 2222.

The angle-limiting portion 2223 further comprises an elastic member 22231 and an angle-limiting member 22232. The elastic element 22231 is positioned at the bottom of the angle-limiting member 22232. A press end 22233 of the angle-limiting member 22232 extends outward from the fixed portion 2221 for user actuation, such that a limit end 22234 of the angle-limiting member 22232 enters the interior of the rotation portion 2222, thereby allowing the angle-limiting member 22232 to rotate together with the rotation portion 2222. When the press end 22233 of the angle-limiting member 22232 is released, the limiting end 22234 is driven by the elastic element 22231 into the space between the fixed portion 2221 and the rotating portion 2222, thereby restricting the rotation of the rotating portion 2222. In other words, in its initial position, the press end 22233 of the angle-limiting member 22232 extends outward from the fixed portion 2221 to facilitate manual actuation. The limit end 22234 of the angle-limiting member 22232 is disposed between the fixed portion 2221 and the rotating portion 2222 to restrict the rotation of the rotating portion 2222. The inner side of the fixed portion 2221 is provided with a plurality of first blocking members 22211. These first blocking members 22211 disposed on the inner side of the sidewall of the fixed portion 2221. Preferably, the first blocking members 22211 are embodied as teeth shape. the inner side of the rotating portion 2222 is provided with a plurality of second blocking members 22221. These second blocking members 22221 are disposed on the inner side of the sidewall of the rotating portion 2222. The angle-limiting member 22232 is embedded within the second blocking members 22221, allowing the angle-limiting member 22232 to rotate together with the rotating portion 2222. Preferably, the angle-limiting member 22232 is embodied as a gear.

The jaw support device 23 further comprises two side support members 231 and a jaw adjustment member 232. The two side support members 231 are respectively disposed on opposite sides of the jaw support device 23. Each of side support members 231 is connected to a respective rotating portion 2222, such that the angle of the jaw support device 23 can be adjusted via rotation of the rotation portions 2222. The two end portions of the jaw adjustment member 232 are respectively connected to the end portions of the two side support members 231. The jaw adjustment member 232 further comprises a support unit 2321 and an adjustment unit 2322. The support unit 2321 is fixed to the adjustment unit 2322. The adjustment unit 2322 is connected to the two side support members 231. The adjustment unit 2322 further comprises an adjustment knob 23221 and two adjustment straps 23222. The two adjustment straps 23222 are movably connected to the adjustment unit 2322 such that the relative positions of the two adjustment straps 23222 can be adjusted via rotation of the adjustment knob 23221, thereby adjusting the width of the jaw support device 23 to conform to the width of the user's jaw. Preferably, the adjustment knob 23221 is provided with a gear. One side of each of the two adjustment straps 23222 is provided with a toothed belt 23223. The two toothed belts 23223 are disposed opposite each other such that they are respectively engaged with the gear of the adjustment knob 23221. In this manner, rotating the adjustment knob 23221 adjusts the width of the jaw support device 23 to accommodate the user's jaw width.

The fixed portion 2221 further comprises a connection portion 22212, which is connected to a side portion of the rear neck support portion 10. Preferably, a connecting strap is used to connect the rear neck support portion 10 to the connection portion 22212, thereby securing the rear neck support portion 10 to the rear neck of the user.

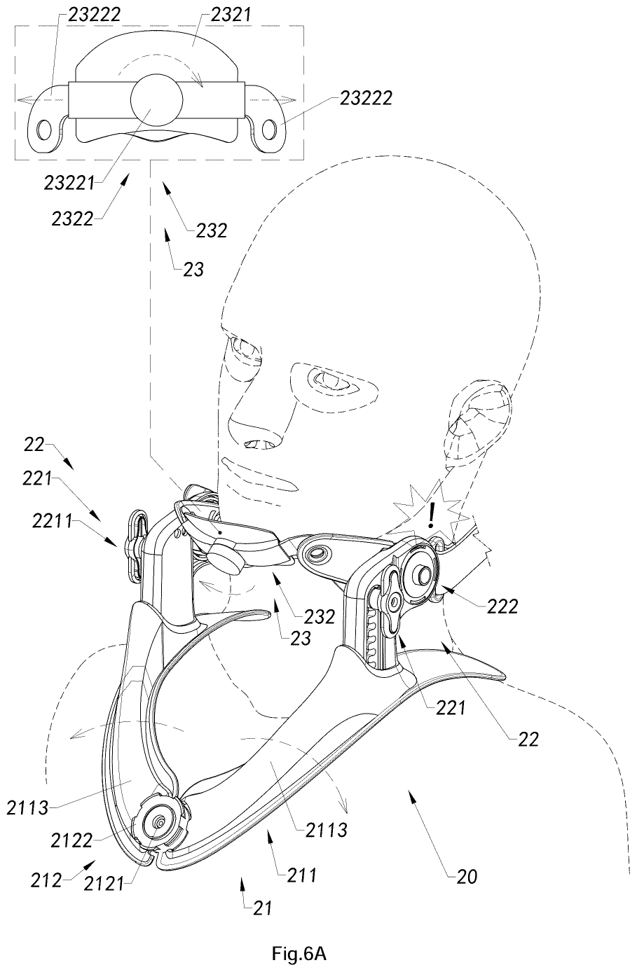

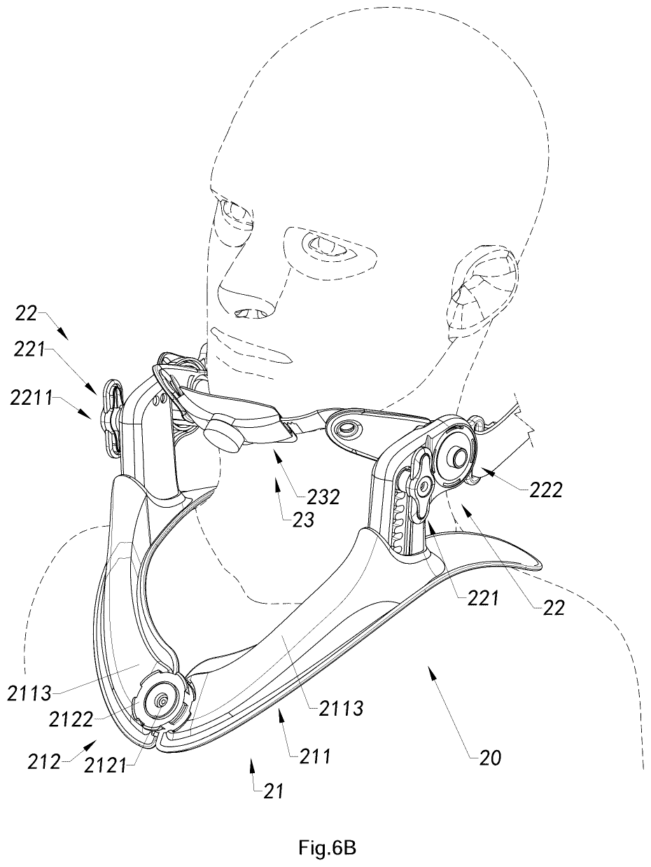

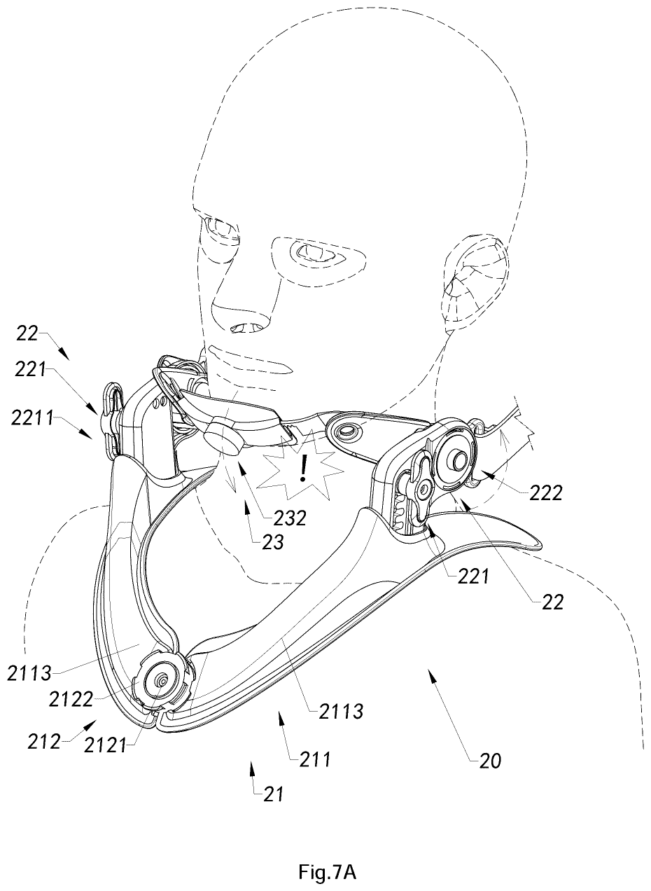

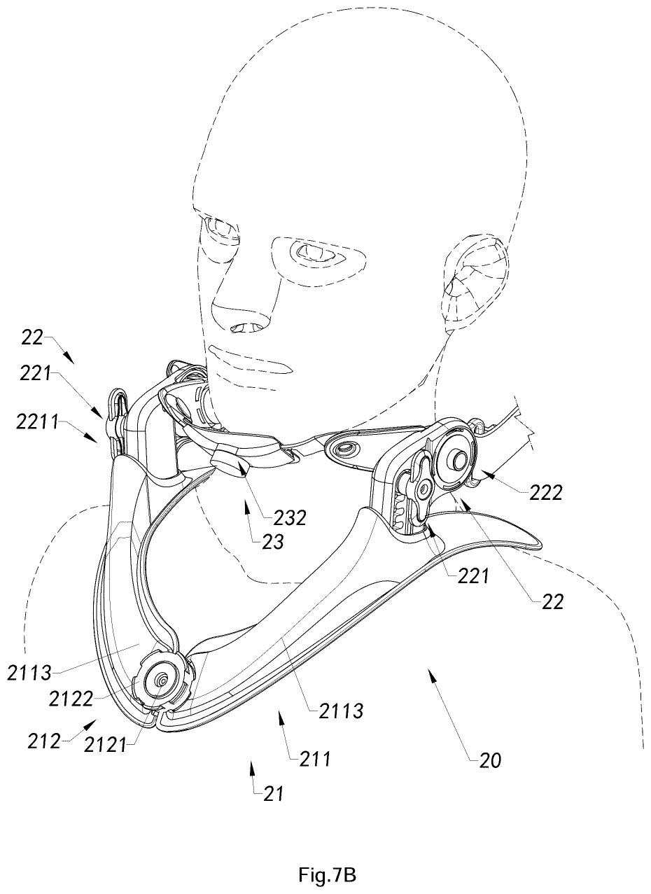

It is worth noting that the present invention provides an adjustment system, as illustrated in FIGS. 6A, 6B, 7A, and 7B. In the adjustment system, the widths of the jaw support device 23 and the adjustable base 21 are separately adjusted to allow the neck brace device of the present invention to adapt the width of the user's neck. Additionally, by adjusting the lengths of the two adjustment devices 22, the distance between the jaw support device 23 and the adjustable base 21 can be varied to match the length of the user's neck.

It is worth mentioning that, to adapt a broader range of users, the inventors of the present invention have discovered that the posture of a user, such as tilting the head upward or downward, is able to affect the apparent length of the neck. In other words, the jaw support device 23 is angle-adjustable and can be tilted upward or downward. Specifically, when the jaw support device 23 is rotated downward to a head-down angle, the neck brace device is suitable for users with shorter necks. Conversely, for users with longer necks, the jaw support device 23 may be rotated upward into a head-raised orientation to adapt longer neck lengths.

The following describes the adjustment system in the context of a user wearing the neck brace device of the present invention. The adjustment platform 20 is placed around the user's neck. During the wearing process, the user's neck first comes into contact with the adjustable base 21. If the user's neck is relatively wide, the two shoulder support members 211 move away from the user's neck. The adjustment section 212 adapts the movement of the two shoulder support members 211 by making corresponding adjustments. In a preferred embodiment of the present invention, the adjustment section 212 increases the distance between the two shoulder support members 211 through rotational movement. When the user's neck moves into contact with the two adjustment mechanisms 22, if the user's neck can continue to move forward, the user's jaw may reach the jaw support device 23 and be supported by it. If the user's neck is obstructed by the two adjustment mechanisms 22 and cannot move forward, the width of the jaw support device 23 must be adjusted to allow the user's neck to continue moving toward the jaw support device 23. In a preferred embodiment of the present invention, the lengths of the two adjustment straps 23222 can be extended by rotating the adjustment knob 23221, enabling the user's neck to continue moving toward the jaw support device 23. Once the user's jaw is supported by the jaw support device 23, the user can further adjust the jaw support device 23 to ensure it conforms to the user's jaw. Specifically, the adjustment knob 23221 is rotated to independently move the two adjustment straps 23222, thereby reducing the distance between the two side support members 231. This adjustment causes the side support members 231 and the jaw adjustment member 232 to conform to the user's jaw. It is worth noting that during the adjustment process to ensure the jaw support device 23 conforms to the user's jaw, the two side support members 231 guide the two adjustment mechanisms 22 to move closer to the user's neck. This movement also drives the two shoulder support members 211 to move closer to the user's neck, ensuring a secure and comfortable fit.

The following describes the adjustment system of the neck brace device through the wearing process of a user. The adjustment platform 20 is worn around the user's neck. During the wearing process, the user's neck engages with the adjustable base 21. If the user's neck is relatively wide, the two shoulder support elements 211 move outward, away from the user's neck. The adjustment portion 212 correspondingly adapts to the movement of the two shoulder support elements 211. In a preferred embodiment of the present invention, the adjustment portion 212 increases the distance between the two shoulder support elements 211 via a rotational mechanism. When the user's neck advances to the two adjustment devices 22, if the neck can continue to move forward, the user's jaw reaches and is supported by the jaw support device 23. If the neck is blocked by the two adjustment devices 22 from moving forward, the width of the jaw support device 23 must be adjusted to allow the neck to continue moving toward the jaw support device 23. In a preferred embodiment of the present invention, this is achieved by rotating the adjustment knob 23221 to respectively extend the lengths of the two adjustment straps 23222, thereby allowing the neck to reach the jaw support device 23. When the user's jaw is supported by the jaw support device 23, the user may further adjust the jaw support device 23 to fit the jaw. Specifically, by rotating the adjustment knob 23221, the two adjustment straps 23222 are moved to reduce the distance between the two side support elements 231, thereby enabling the side support elements 231 and the jaw adjustment element 232 to closely conform to the user's jaw. It is worth mentioning that during the adjustment process in which the jaw support device 23 conforms to the user's jaw, the two side support elements 231 guide the two adjustment devices 22 toward the direction of the user's neck, thereby driving the two shoulder support elements 211 to also move toward the user's neck, achieving an overall conformance.

If the user's neck is relatively short and the jaw support device 23 fails to provide adequate support to the user's jaw, the height of the jaw support device 23 may be lowered by operating the two knob elements 2211 to respectively adjust the two adjustment devices 22. Additionally, the user may press the pressing end 22233, such that the angle-limiting end 22234 enters the rotating portion 2222, thereby allowing the jaw support device 23 to rotate downward to a position where it can support the user's jaw. If the user's neck is relatively long, the jaw support device 23 may be elevated by adjusting the two adjustment devices 22. The user may further adjust the upward tilt angle of the jaw support device 23 to increase the height, thereby better accommodating the length of the user's neck.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and are subject to change without departure from such principles. Therefore, this invention includes all modifications encompassed within the spirit and scope of the following claims.

What is claimed is:

1. A neck brace device for wearing on a user's neck, comprising:
an adjustment platform for surrounding the neck of the user, wherein said adjust platform further comprises a jaw support device, wherein said jaw support device is disposed on the top portion of said adjustment platform for supporting the jaw of the user, wherein said jaw support device is adjustable in a horizontal direction to adapt the width of the user's jaw.

2. The neck brace device, as recited in claim 1, wherein said neck brace device further comprises an adjustable base disposed below the jaw support device wherein said adjustable base is adjusted in a horizontal direction to adapt to the width of the user's neck, wherein horizontal adjustment of said jaw support device to reduce said jaw support device's width guides corresponding adjustment of said adjustable base to reduce the width of said adjustable base.

3. The neck brace device, as recited in claim 2, wherein said adjustable base further comprises two shoulder support members connected adjustably, wherein a wearing cavity is defined between said two shoulder support members to receive the user's neck, wherein when said jaw support device is adjusted in a horizontal direction to reduce said jaw support device's width, said two shoulder support members are guided to move toward each other to reduce said width of the wearing cavity.

4. The neck brace device, as recited in claim 3, wherein said two shoulder support members are pivotally connected, wherein when said jaw support device is adjusted in a horizontal direction to reduce the width of the jaw support device, said two shoulder support members are guided to pivot toward the user's neck.

5. The neck brace device, as recited in claim 3, wherein said adjustment platform further comprises two adjustment devices, wherein said two adjustment devices are separately disposed between said tow shoulder support members and said jaw support device, configured to adjust a distance between said two shoulder support members and said jaw support device and an angle of said jaw support device, wherein the jaw support device is rotatable toward said two shoulder support members to support said user's jaw in a head-down posture.

6. The neck brace device, as recited in claim 5, wherein the jaw support device further comprises:
two side support members, said two side support members being separately rotatably connected to said two adjustment devices, such that said two adjustment devices adjust and angel of said jaw support device; and
a jaw adjustment member, opposite ends of said jaw adjustment member being connected to respective ends of said two side support members, said jaw adjustment member being configured to be adjusted in a horizontal direction to accommodate a width of the user's jaw, wherein, by adjusting said jaw adjustment member to reduce said jaw adjustment member's width, said two side support members are driven to move toward the user's neck, thereby driving said two adjustment devices to move toward said user's neck, and further causing said two shoulder support members to move toward the user's neck.

7. The neck brace as recited in claim 6, wherein the jaw adjustment member further comprises a support unit and an adjustment unit, wherein said adjustment unit is movably connected to said support unit, wherein said support unit is configured to conform to said user's jaw, and wherein the length of said adjustment unit is adjustable to adapt the width of the user's jaw.

8. The neck brace, as recited in claim 7, wherein said adjustment unit further comprises:
an adjustment knob, wherein one end of said adjustment knob is provided with a gear;
two adjustment straps, each of said adjustment straps having a toothed side with a plurality of teeth formed thereon to define a toothed belt, wherein said adjustment knob is disposed between said two adjustment straps, wherein said toothed sides of said toothed sides of said two adjustment straps face each other, and wherein said gear of said adjustment knob is engaged with said toothed belts of said two adjustment straps, such that the width of said adjustment unit is adjustable by changing the relative positions of said two adjustment straps.

9. The neck brace, as recited in claim 7, wherein said adjustment unit is selected from a parallel displacement mechanism and an elastic device.

10. The neck brace, as recited in claim 5, wherein the adjustable base further comprises an adjustment portion, said two shoulder support members are separately connected to the adjustment portion, wherein said adjustment portion further comprises a rotation shaft and a coupling member, said coupling member is sleeved over said rotation shaft such that said two shoulder support members are rotatable about said rotation shaft.

11. The neck brace, as recited in claim 5, wherein said adjustable base further comprises an adjustment portion, said two shoulder support members are separately rotatably connected to said adjustment portion, wherein said adjustment portion further comprises two rotation shafts and two rotation shafts and two coupling members, each of said coupling members is sleeved over a respective rotation shaft such that said two shoulder support members are rotatably connected to said adjustment portion.

12. The neck brace, as recited in claim 10, wherein said rotation shaft comprises a first limiting member disposed on an outer side of a bottom portion of said first limiting member, and said coupling member comprises a second limiting member disposed on an outer side of a bottom portion of said coupling member, wherein a clearance is defined between two ends of said first limiting member and said second limiting member to form an angle-limiting mechanism that restricts a rotation angle of said two shoulder adjustment devices.

13. The neck brace, as recited in claim 5, wherein said adjustment base further comprises an adjustment portion, where said adjustment portion is selected from a parallel linkage mechanism, a ratchet mechanism, and an elastic band.

14. The neck brace, as recited in claim 5, wherein said adjustment device further comprises a distance adjustment device and an angle adjustment device, wherein said angle adjustment device is configured to adjust an angle of said jaw support device, and wherein said angle adjustment device locks said jaw support device in position to prevent rotation of said jaw support device during vertical adjustment by said distance adjustment device.

15. The neck brace as recited in claim 14, wherein said distance adjustment device further comprises a knob, an adjustment rack, and adjustment bracket, and an adjustment device housing, wherein said knob and said adjustment rack are mounted on said adjustment bracket, wherein said adjustment bracket is mounted on said mounting bracket, and wherein said adjustment bracket is adapted within said adjustment device housing.

16. The neck brace, as recited in claim 14, wherein said angle adjustment device, further comprises a fixed portion, a rotation portion, and an angle-limiting portion, wherein said fixed portion and said rotation portion are disposed opposite each other, wherein one end of said angle-limiting portion is movable between the interior of said fixed portion and the interior of the rotation portion, wherein when the said one end of said angle-limiting portion is positioned between said fixed portion and said rotation portion, said angel adjustment device is in a locked state such that the position of said jaw support device is fixed, wherein when said one end of said angle-limiting portion is positioned within said rotation portion, said angle adjustment device is an unlocked state, allowing said jaw support device to be adjusted to a suitable angle.

17. The neck brace, as recited in claim 16, wherein the angle-limiting portion comprises an angle-limiting member and an elastic element, wherein the elastic element is disposed within the rotation portion, and a bottom end of the angle-limiting member is in contact with said elastic element, wherein when said elastic element is not compressed, the angle-limiting member is positioned between the fixed portion and the rotation portion to prevent said rotation portion from rotating, wherein when said elastic element is compressed, said angle-limiting member is displaced into said rotation portion to allow said rotation portion to move.

18. The neck brace, as recited in claim 5, wherein the neck brace further comprises a rear neck support portion, wherein said rear neck support portion is detachably connected to said two adjustment devices to conform to a rear neck of the user by adjusting said rear neck support portion.
